(19) United States
(12) Patent Application Publication
(10) Pub. No.: US 2015/0198600 A1
(43) Pub. Date: Jul. 16, 2015

(54) Autoantibody Profiles in the Early Detection and Diagnosis of Cancer

(71) Applicant: Rowan University, Glassboro, NJ (US)

(72) Inventors: Robert G. Nagele, Turnersville, NJ (US); Eric P. Nagele, Turnersville, NJ (US)

(21) Appl. No.: 14/417,704

(22) PCT Filed: Jul. 26, 2013
(86) PCT No.: PCT/US13/52349

§ 371 Date: Jan. 27, 2015

Related U.S. Application Data
(60) Provisional application No. 61/676,560, filed on Jul. 27, 2012.

(51) Int. Cl. G01N 33/574

(52) U.S. Cl. CPC G01N 33/57484

(57) ABSTRACT

The present invention provides methods, compositions and kits for the detection of cancer diagnostic biomarkers, for the diagnosis of cancer, for the identification of a subject at risk for developing cancer, and for the generation of patient-specific cancer diagnostic biomarker profiles.

### CROSS-REFERENCE TO RELATED APPLICATION

This application claims priority of U.S. Application Ser. No. 61/676,560 filed Jul. 27, 2012, the disclosure of which is incorporated herein in its entirety.

### TECHNICAL FIELD

This invention relates to the use protein microarray technology to identify specific subsets of antibodies in human sera that are useful for the early detection and diagnosis of cancer. One or more microarrays can be used to identify either a specific cancer or a multi-cancer early detection and diagnostic assay. Serum from human blood samples is used to probe these custom microarrays, and they can be evaluated to reveal specific patterns of autoantibody reactivity that are indicative of existing cancers and thus will allow for diagnosis and early detection of many cancer types either individually or simultaneously.

### BACKGROUND OF THE INVENTION

Millions of people around the world are at risk for or are currently suffering from cancer, one of the leading causes of mortality throughout the world. Early detection of cancers can significantly improve the treatment and survival rates of cancer patients. As tumors develop, the cells, tissues, and organs can increase or decrease their release of certain chemicals, including protein secretory and breakdown products in the circulatory system from dead and dying cells. Some of these products that are released and detectable in the blood have already proven useful as biomarkers and have been approved for use by the FDA. A well known example is the PSA (prostate specific antigen) test for prostate cancer detection. A total PSA level of 4 ng/mL is generally considered as a normal threshold. However, when this value exceeds 10 ng/mL, the chances of prostate malignancy is increased substantially.

Until effective therapeutic treatments are developed to prevent, treat, and cure cancer, the best means of reducing mortality and morbidity in a disease of this complexity is early detection and diagnosis. In the major solid cancer types such as lung, breast, colon, and prostate, the long-term survival rates drop precipitously once the cancer has advanced to the point where metastasis has occurred. In addition, diagnostic measurement of cancer disease progression is essential to successful disease management. For these reasons, the development of new and effective biomarkers for cancer detection and diagnosis is central to the cancer problem. Biomarkers are not only useful in following the course of cancer, but also for evaluating which therapeutic strategies are most effective for a particular type of cancer, as well as determining long-term susceptibility to cancer or its recurrence.

Studies on cancer patients have shown such patients sometimes make autoantibodies against their own malignant cells and tissues, as part of an immune response against their cancers. For example, Clausen et al. (2010) have developed a “functional protein” microarray to detect autoantibodies in prostate cancer serum samples. By identifying the antigens to which these autoantibodies are raised, these autoantibodies can be used as biomarkers of disease. Although more commonly linked to autoimmune diseases, the immune system also produces autoantibodies in response to other diseases, including cancer, due to pathological changes that occur during the course of the disease.

These workers developed a microarray of 925 proteins, and then used blood samples to test arrays. They compared the results from 73 samples from patients diagnosed with prostate cancer to 60 samples from a control group of cancer-free individuals to find proteins on the arrays that were bound by autoantibodies present in the blood samples. Panels of up to 15 biomarkers were identified that distinguished prostate cancer from both benign prostate disease and healthy tissue (Reference: Clausen H, et al. Cancer Biomarkers Defined by Autoantibody Signatures to Aberrant O-Glycopeptide Epitopes. Cancer Research, Feb. 15, 2010. DOI 10.1158/0008-5472. CAN-09-2893).

To our knowledge, there is no effective and sensitive blood test for the early detection or accurate diagnosis of most cancers. There is also no one blood test that has the potential to perform this function for multiple cancers simultaneously. Additional techniques, such as CT, MRI, and X-ray may be used to identify abnormalities or suspect masses, but rarely do they detect any abnormality that can be directly related to the onset of cancer. There are also no laboratory tests utilizing blood, cerebrospinal fluid, or urine samples that have proven to be effective in primary diagnosis or confirmation of cancer.

Thus, there is a need for an accurate, relatively non-invasive, and affordable diagnostic test for cancer, especially one that can identify the condition at an early stage of the disease, even before significant physical symptoms are expressed. This is particularly true given widespread recognition that early detection facilitating early treatment helps to slow the progression of the disease, minimize symptoms, and improve the overall quality of life. The present invention provides for new diagnostic tests that will fill this void while also providing an inexpensive multi-disease assay that can be readily incorporated into routine health care.

### BRIEF SUMMARY OF THE INVENTION

At least one aspect of the present invention relates to screening systems for individual cancers such as, for example, Breast, Endometrial, Kidney, Ovarian, Cervical, Testicular, Lung, Prostate, Colorectal, Oral, Stomach, Esophageal, Thyroid, Pancreatic and Bladder Cancers and to a screening system for the simultaneous early detection and diagnosis of multiple cancers on the same protein microarray platform.

In one embodiment, the present invention provides a method for detecting cancer diagnostic biomarkers in a subject in need of such detection comprising obtaining an immunoglobulin-containing biological sample from the subject, and performing an assay to determine the presence or absence of one or more cancer diagnostic biomarkers in the biological sample.

In another embodiment, the present invention provides a method for diagnosing cancers of Breast, Endometrial, Kidney, Ovarian, Cervical, Testicular, Lung, Prostate, Colorectal, Oral, Stomach, Esophageal, Thyroid, Pancreatic and Bladder in a subject in need of such diagnosis comprising obtaining an immunoglobulin-containing biological sample from the subject, performing an assay to determine the presence or absence of one or more specific diagnostic biomarkers of such cancers in the biological sample, and diagnosing the type of cancer if one or more of the cancer diagnostic biomarkers are present.

In another embodiment, the present invention provides a method of identifying a subject at risk for developing cancer comprising obtaining an immunoglobulin-containing biological sample from the subject, performing an assay to determine the presence or absence of one or more cancer diagnostic biomarkers in the biological sample, and identifying the subject as at risk for developing cancer if one or more of the cancer diagnostic biomarkers is present.

In another embodiment, the present invention provides a method of generating a patient-specific cancer diagnostic biomarker profile comprising obtaining an immunoglobulin-containing biological sample from a patient, performing an assay to determine the presence or absence of one or more cancer diagnostic biomarkers in the biological sample, and generating a patient-specific cancer diagnostic biomarker profile of the cancer diagnostic biomarkers present in the sample.

In yet another embodiment, the present invention provides a substrate on which one or more antigens that are specific for a cancer diagnostic biomarker are immobilized.

In yet another embodiment, the present invention provides a substrate on which one or more antigens specific for a diagnostic biomarker for cancers of Breast, Endometrial, Kidney, Ovarian, Cervical, Testicular, Lung, Prostate, Colorectal, Oral, Stomach, Esophageal, Thyroid, Pancreatic and Bladder are immobilized.

The present invention provides, in another embodiment, a microarray comprising a substrate on which one or more antigens that are specific for a diagnostic biomarker of are immobilized.

In a further embodiment, the present invention provides a kit for detecting cancer-specific antibodies, particularly for cancers of Breast, Endometrial, Kidney, Ovarian, Cervical, Testicular, Lung, Prostate, Colorectal, Oral, Stomach, Esophageal, Thyroid, Pancreatic and Bladder.

The details of one or more embodiments of the invention are set forth in the description below. Other features, objects, and advantages of the invention will be apparent from the description and from the claims.

### BRIEF DESCRIPTION OF THE DRAWINGS

FIG. 1 shows a schematic illustration of a diagnostic test of the invention.

FIG. 2 shows a schematic of Protein In Situ Arrays (PISA).

FIG. 3 shows a schematic of nucleic acid programmable arrays (NAPPA).

FIG. 4 shows the reproducibility of DAPA in multiple copies.

FIG. 5 shows a method of making and using a protein array.

FIG. 6 shows a segment of the list of diagnostic proteins.

FIG. 7 is a graphical representation of the PAM significance list.

FIG. 8 is a list of antibody importance.

### DETAILED DESCRIPTION OF THE INVENTION

For the first time, the present inventors have discovered that the presence of disease is accompanied by the production of a large number of auto-reactive antibodies useful for cancer diagnostics. Accordingly, they have used specific autoantibodies as biomarkers for different cancer subtypes and incorporate them into the microarray platform for the creation of multi-diagnostic tools.

The present inventors have shown that autoantibodies (self-reactive antibodies) are both abundant and ubiquitous in human sera, regardless of age or the presence or absence of disease (Levin et al., 2010; Nagele et al., 2011). Some are brain-reactive, others are reactive to targets in other organs throughout the body. The underlying reason for their presence and abundance in human sera, especially in younger otherwise healthy individuals, is not known, but their presence in all human sera examined thus far (n>200) is irrefutable. Although some autoantibodies may be vestiges of past diseases and immunological activity, it is clear, especially in the elderly, that many autoantibodies are also present in the blood as a result of existing or ongoing diseases.

As shown in the present invention, it is this latter group that is useful for the early detection and diagnosis of existing diseases like cancer. The presence of active disease (both long- and short-term diseases) including cancers causes the production and release of cellular products as a result of cell damage related to ongoing pathology, some of which are both cell type- and organ-specific. In the case of cancers, the forming hypoxic and later necrotic cores of developing tumors provides a source of cell breakdown products. For unknown reasons, these released cellular products (many of which are proteins), their break-down fragments and disease-related post-translation modifications spill into the blood and lymph circulation, act as antigens and secondarily elicit an immune response. This immune response leads to the production and appearance of a relatively large number of self-reactive autoantibodies in the blood. Since cells throughout the body share a vast number of proteins in common, only a relatively small subset of autoantibodies will be specifically reactive to the cells, tissues and organs involved in the disease. Nevertheless, the end result is the generation of a disease-specific autoantibody profile in the blood that is characteristic for each disease and the specific cell types involved, much like a fingerprint can be used to identify an individual.

In addition, in individuals with concurrent diseases, the specific pattern of autoantibodies in these individuals will reflect each of these concurrent, ongoing disease processes. Recognition of these individual disease autoantibody “fingerprints” of course depends on one's ability to “dissect out” and recognize individual disease-specific autoantibody profiles. This we have already accomplished for Alzheimer's disease, Parkinson's disease, and breast cancer.

The present inventors hereby show how autoantibody profiles in the blood can be effectively used to accurately diagnose cancers based on the identification of disease-specific autoantibody and target antigen profiles. In at least one aspect of the present invention, these autoantibodies can be used to develop screening systems for individual cancers such as, for example, Breast, Endometrial, Kidney, Ovarian, Cervical, Testicular, Lung, Prostate, Colorectal, Oral, Stomach, Esophageal, Thyroid, Pancreatic and Bladder Cancers.

In another aspect of the invention, the inventors develop a screening system for the simultaneous early detection and diagnosis of multiple cancers on the same protein microarray platform.

In at least one embodiment, protein microarrays containing thousands of full-sized or nearly full-sized human proteins spotted onto a single specimen slide are described to identify all of the autoantibodies in the blood that were reactive to the antigen targets presented on the microarray. In another embodiment, the resulting data are subjected to sophisticated and unbiased, computer-assisted comparative analysis, with the goal of identifying the subset of specific autoantibodies and targets that are useful in detecting and diagnosing specific cancers and distinguishing them from other diseases and cancers.

In another embodiment, the identified disease-specific subsets are analyzed with computer programs that identify those autoantibodies and protein targets that have the greatest value in detection and determining the diagnosis. In yet another embodiment, such analysis is performed, with the goal of determining the minimum number of protein targets required for maximum detection and diagnostic accuracy, sensitivity and specificity.

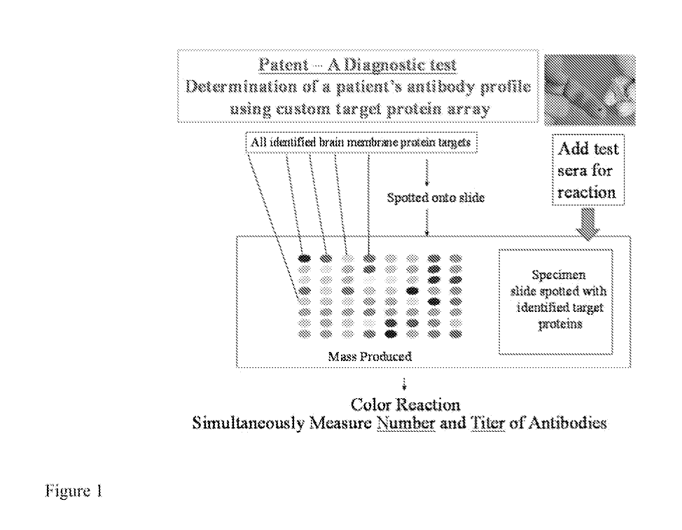

In one embodiment, the useful protein targets identified by the aforementioned methodology are spotted onto specially treated slides to form a protein microarray and are further used for early detection and diagnosis of breast cancer. A schematic of the diagnostic test is shown in FIG. 1 indicating that autoantibodies are powerful biomarkers for disease detection.

In another aspect of the present invention, protein target microarrays that contain the identified and selected disease-relevant target antigens are constructed that can be used to detect and diagnose multiple cancers after probing these arrays with autoantibodies that are present in a sample of human serum as small as a single drop. Accordingly, in at least one feature of the present invention, diagnostic systems are described that can perform the screening in a non-invasive and practical manner only with a single drop of blood from a single finger stick or blood draw tube.

The presently described method and system has great sensitivity, specificity and reliability, can be easily accomplished by individuals with little or no special skills or training, or be readily automated.

In more detail aspect of the present invention, diagnostic kits are described which uses target protein (antigen) microarrays for detecting and monitoring the spectrum (number and quantity) of autoantibodies in human sera and for the discovery of proteins and antibodies implicated in disease and the rapid detection or diagnosis of disease. According to this aspect of the invention, unique opportunities are provided to health care practitioners to perform disease risk assessment, disease diagnostics, biomarker discovery, staging of diseases in a patient, predicting disease recurrence and drug target discovery.

In another embodiment of the present invention, the protein microarrays described herein can be used to detect new autoantibodies that are linked to specific diseases and new antigen targets. In yet another embodiment, thousands of proteins and/or antibodies can be incorporated onto a single slide for analysis, with the potential of detecting thousands of potential antibodies that may exist across the human population in a wide variety of diseases.

In another aspect of the present invention, quantitative detection of biomarkers is described. Accordingly, the present method reveals the relative titer or concentration of autoantibodies in the serum. This is particularly important since, in many diseases such as in allergies, it is not the presence of the antibody that is critical, rather the fact that it is overproduced by the body and its overabundant that prompts clinically problematic and pathological growth.

Those of ordinary skill in the art can appreciate other uses of the present methodology. Accordingly the present inventors contemplate other uses such as simultaneous early detection and assessment of single or multiple cancers, simultaneous diagnosis of single or multiple cancers, staging of cancers, determining the response of a patient to therapy and finally employing the methodology as an assessment tool for clinical trials monitoring of the efficacy of certain drugs in treatment of cancers.

In another embodiment, cancer diagnostic biomarkers are defined herein as antibodies, including for example autoantibodies that specifically bind to protein antigens and are diagnostic indicators that can be used to differentiate cancer subtypes from control subjects without the respective cancer condition. In one embodiment, approximately 20-30 autoantibodies and their targets have been identified for breast cancer.

Experimental data supporting and illustrating this approach are presented below, using breast cancer data as an example.

The term “protein antigens” as used herein includes protein and peptide antigens. Protein antigens that have been identified as capable of being specifically bound by the cancer diagnostic biomarkers are set forth in the following Table 1. The protein antigens in Table 1 are identified by art-accepted names as well as database identification numbers. The database identification numbers refer to the publically available protein databases of the National Center for Biotechnology Information (NCBI), which are well-known and accessible to those of ordinary skill in the art.

TABLE 1

| | id | Name | BC score | CON score |
|---|---|---|---|---|
| 10 | BC016380 | cDNA clone MGC: 27376 IMAGE: 4688477, complete cds | 0.9055 | −0.4528 |
| 11 | NM_00314 | Tripartite motif-containing 21 (TRIM21) | 0.668 | −0.334 |
| 12 | NM_03234 | nudix (nucleoside diphosphate linked moiety X)-type motif 16- | −0.6619 | 0.331 |
| 13 | BC015833 | cDNA clone MGC: 27152 IMAGE: 4691630, complete cds | 0.6013 | −0.3007 |
| 14 | PHR5001 | Recombinant human CTLA-4/Fc | 0.4405 | −0.2203 |
| 15 | BC030984 | cDNA clone MG: 32664 IMAGE: 4701898, complete cds | 0.4005 | −0.2003 |
| 16 | BC014271 | endoglin (Osler-Rendu-Weber syndrome 1) (ENG) | 0.3763 | −0.1881 |
| 17 | BCO20233 | cDNA clone MGC: 31936 IMAGE: 4765518, complete cds | 0.347 | −0.1735 |
| 18 | 8C001304 | piccolo (presynaptic cytomatrix protein) (POLO) | 0.2567 | −0.1283 |
| 19 | PH 1705 | fms-related tyrosine kinase 3 ligand (FLT3LG) | −0.2382 | 0.1191 |
| 20 | BC032451 | cDNA clone MG: 40426 IMAGE: 5178085, complete cds | 0.1976 | −0.0988 |
| 21 | BCO22362 | cDNA clone MG: 23888 IMAGE: 4704496, complete cds | −0.1894 | 0.0947 |
| 22 | NM_00498 | LIM and senescent cell antigen-like-containing domain protein | −0.1884 | 0.0942 |
| 23 | BC047536 | sciellin (SCEL) | 0.1752 | −0.0876 |
| 24 | BC053656 | EGF-like repeats and discoidin 1-like domains 3 (EDIL3) | 0.1641 | −0.0821 |
| 25 | 8C000306 | hydroxyacyl-Coenzyme A dehydrogenase (HADH) | 0.1567 | −0.0784 |
| 26 | BC030813 | cDNA clone MGC: 22645 IMAGE: 4700961, complete cds | 0.1327 | −0.0664 |
| 27 | NM_02121 | chromosome 9 open reading frame 80 (C9or1B80) | 0.1229 | −0.0615 |
| 28 | BC014991 | N-methylpurine-DNA glycosylase (MPG) | −0.1157 | 0.0578 |
| 29 | BC000468 | ubiquitin-conjugating enzyme E2 variant 1 (UBE2V1) | 0.1058 | −0.0529 |
| 30 | NM_00679 | myotilin (MYOT) | 0.096 | −0.048 |
| 31 | NM_01512 | FCH domain only 1 (FCH01) | −0.0953 | 0.0476 |
| 32 | BC019015 | mediator complex subunit 29 (MED29) | 0.0807 | −0.0404 |
| 33 | BC009993 | chromosome 3 open reading frame 37 (C3orf37) | 0.0742 | −0.0371 |
| 34 | NM_17354 | Tripartite motif-containing 65 (TRIM65) | −0.07 | 0.035 |
| 35 | NM_01324 | chromosome 16 open reading frame 80 (C16or180) | 0.0226 | −0.0113 |
| 36 | BC051762 | Uncharacterized protein C20orf96 | 0.0189 | −0.0095 |
| 37 | NM_00116 | baculoviral IAP repeat-containing 4 (BIRC4) | 0.0168 | −0.0084 |
| 38 | NM_01411 | protein phosphatase 1, regulatory (inhibitor) subunit 8 (PPP1I | −0.0153 | 0.0076 |
| 39 | NM_15287 | Tumor necrosis factor receptor superfamily member 6 | 0.0146 | −0.0073 |

Thus in one embodiment, the present invention provides a method for detecting cancer diagnostic biomarkers in a subject in need of such detection comprising obtaining an immunoglobulin-containing biological sample from the subject, and performing an assay to determine the presence or absence of one or more cancer diagnostic biomarkers in the biological sample.

In another embodiment, the present invention provides a method for diagnosing cancer in a subject in need of such diagnosis comprising obtaining an immunoglobulin-containing biological sample from the subject, performing an assay to determine the presence or absence of one or more cancer diagnostic biomarkers in the biological sample, and diagnosing cancer if one or more cancer diagnostic biomarkers are present.

In a preferred embodiment, the subject is a human subject.

In a preferred embodiment of the invention, the immunoglobulin-containing biological sample is serum, plasma, whole blood, CSF, saliva, or sputum. A blood sample may be obtained by methods known in the art including venipuncture or a finger stick. CSF may be obtained by methods known in the art including a lumbar spinal tap. Serum and plasma samples may be obtained by centrifugation methods known in the art. Sputum and saliva samples may be collected by methods known in the art. The biological samples may be diluted with a suitable buffer before conducting the assay. In a preferred embodiment, the biological sample is serum, plasma or whole blood.

Assays to determine the presence or absence of one or more cancer diagnostic biomarkers in the biological sample are performed by contacting the sample with one or more antigens that are specific to a cancer type diagnostic biomarker under conditions that allow an immunocomplex of the antigen and the antibody to form, and detecting the presence of the immunocomplex

An antigen may comprise a protein antigen of Table 1 or a polypeptide or peptide fragment thereof containing one or more epitopes recognized by the cancer diagnostic biomarker, or an epitope peptidomimetic that is recognized by the cancer diagnostic biomarker. Peptidomimetics include, for example, D-peptides, peptoids, and β-peptides. The antigens may be purified from natural sources, or produced recombinantly or synthetically by methods known in the art, and may be in the form of fusion proteins. The antigens may be produced in vitro using cell-free translation systems. In one preferred embodiment, the antigens are produced in a mammalian or insect expression system to ensure correct folding and function. All of these methods may be automated for high throughput production.

Assays and conditions for the detection oft immunocomplexes are known to those of skill in the art. Such assays include, for example, competition assays, direct reaction assays and sandwich-type assays. The assays may be quantitative or qualitative. In one preferred embodiment, the assay utilizes a solid phase or substrate to which the antigens are directly or indirectly attached, such as a microtiter or microassay plate, slide, magnetic bead, non-magnetic bead, column, matrix, membrane, or sheet, and may be composed of a synthetic material such as polystyrene, polyvinyl chloride, polyamide, or other synthetic polymers, natural polymers such as cellulose, derivatized natural polymers such as cellulose acetate or nitrocellulose, and glass, for example glass fibers. The substrate preferably comprises a plurality of individually addressable antigens immobilized on the surface. The individually addressable antigens are preferably immobilized on the surface to form an array. The substrates may be used in suitable shapes, such as films, sheets, or plates, or may be coated onto or bonded or laminated to appropriate inert carriers, such as paper, glass, plastic films, or fabrics. In a preferred embodiment, the substrate is a slide or a bead.

Methods for attaching the antigens to the support or substrate are known in the art and include covalent and noncovalent interactions. For example, diffusion of applied proteins into a porous surface such a hydrogel allows noncovalent binding of unmodified protein within hydrogel structures. Covalent coupling methods provide a stable linkage and may be applied to a range of proteins. Biological capture methods utilising a tag (e.g., hexahistidine/Ni-NTA or biotin/avidin) on the protein and a partner reagent immobilized on the surface of the substrate provide a stable linkage and bind the protein specifically and in reproducible orientation.

In one preferred embodiment, the antigens are coated or spotted onto the support or substrate such as chemically derivatized glass.

In one preferred embodiment the antigens are provided in the form of an array, and preferably a microarray. Protein microarrays are known in the art and reviewed for example by fall et al. (2007) Mech Ageing Dev 128:161-167 and Stoevesandt et al (2009) Expert Rev Proteomics 6:145-157, the disclosures of which are incorporated herein by reference. Microarrays may be prepared by immobilizing purified antigens on a substrate such as a treated microscope slide using a contact spotter or a non-contact microarrayer. Microarrays may also be produced through in situ cell-free synthesis directly from corresponding DNA arrays.

Suitable methods for external production and purification of antigens to be spotted on arrays include expression in bacteria, as disclosed for example by Venkataram et al. (2008) Biochemistry 47:6590-6601, in yeast, as disclosed for example by Li et al. (2007) Appl Biochem Biotechnol. 142:105-124, in insect cells, as disclosed for example by Altman et al. (1999) Glycoconj J 16:109-123, and in mammalian cells, as disclosed for example by Spampinato et al. (2007) Curr Drug Targets 8:137-146.

Suitable methods for in situ (“on-chip”) protein production are disclosed, for example, by Ramachandran et al. (2006) Methods Mol. Biol 2328:1-14 and He et al. (2008) Curr. Opin Biotechnol 19:4-9.

Other methods by which proteins are simultaneously expressed and immobilized in parallel on an array surface are also known in the art and may be used in accordance with the present invention. For example, in the Protein In Situ Arrays (PISA) method (He et al. (2001) Nucleic Acids Res 29:e73), proteins are made directly from DNA, either in solution or immobilized, and become attached to the array surface as they are made through recognition of a tag sequence. The proteins are expressed in parallel in vitro utilizing a cell free system, commonly rabbit reticulocyte or E. coli S30, to perform coupled transcription and translation. In this method, protein expression is performed on a surface which is precoated with an immobilizing agent capable of binding to the tag. Thus after each protein is translated, it becomes fixed simultaneously and specifically to the adjacent surface, while the other materials can subsequently be washed away. Microarrays are produced directly onto glass slides, either by mixing the DNA with the cell free lysate system before spotting or by a multiple spotting technique (MIST) in which DNA is spotted first followed by the expression system.

In the system known as Nucleic Acid Programmable Protein Array (NAPPA) (Ramachandran et al. (2004) Science 305:86-90), transcription and translation from an immobilized (as opposed to a solution) DNA template allow conversion of DNA arrays to protein arrays. In this method, biotinylated cDNA plasmids encoding the proteins as GST fusions are printed onto an avidin-coated slide, together with an anti-GST antibody acting as the capture entity. The cDNA array is then covered with rabbit reticulocyte lysate to express the proteins, which become trapped by the antibody adjacent to each DNA spot, the proteins thereby becoming immobilized with the same layout as the cDNA. This technology generates a protein array in which the immobilized proteins are present together with DNA and a capture agent.

Another suitable method for generating a protein array is the DNA Array to Protein Array (DAPA) method. This method for in situ protein arraying uses an immobilized DNA array as the template to generate ‘pure’ protein arrays on a separate surface from the DNA, and also can produce multiple copies of a protein array from the same DNA template (He et al. (2008) Nature Methods, 5:175-7). Cell-free protein synthesis is performed in a membrane held between two surfaces (e.g., glass slides), one of which is arrayed with DNA molecules while the other surface carries a specific reagent to capture the translated proteins. Individual, tagged proteins are synthesized in parallel from the arrayed DNA, diffuse across the gap and are subsequently immobilized through interaction with the tag-capturing reagent on the opposite surface to form a protein array. Discrete spots which accurately reflect the DNA in position and quantity are produced. Replicate copies of the protein array can be obtained by reuse of the DNA.

Array fabrication methods include robotic contact printing, ink-jetting, piezoelectric spotting and photolithography. For example, purified antigens of the invention that are produced and purified externally may be spotted onto a microarray substrate using a flexible protein microarray inkjet printing system (e.g., ArrayJet, Roslin, Scotland, UK) to provide high quality protein microarray production. The precise rows and columns of antigens may be converted to detectable spots denoting both the presence and amount of diagnostic biomarkers that have been bound.

The production of the microarrays is preferably performed with commercially available printing buffers designed to maintain the three-dimensional shape of the antigens. In one preferred embodiment, the substrate for the microarray is a nitrocellulose-coated glass slide.

The assays are performed by methods known in the art in which the one or more antigens are contacted with the biological sample under conditions that allow the formation of an immunocomplex of an antigen and an antibody, and detecting the immunocomplex. The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software.

In one preferred embodiment of the present invention, fluorescence labeling and detection methods are used to detect the immunocomplexes. Commercially available slide scanners (e.g. the Genepix 4000B slide scanner (Molecular Devices, Inc.) with associated analytical software may be used. In one preferred embodiment, the immunocomplex is probed with fluorescent-labeled (e.g., Alexa-Fluor (Invitrogen)) anti-human antibody and the intensity of fluorescence at each protein spot is measured using a microarray scanner. Commercially available software (e.g. GenePix Pro 5.0 software (Axon instruments)) may be used to extract the net median pixel intensities for individual features from the digital images produced by the scanner. Data may be normalized by comparing median values of multiple identical control spots in different regions of the same array.

Detection of diagnostic immunocomplexes is indicative of the presence of cancer diagnostic biomarkers in the biological sample, and thus a positive diagnosis of cancer.

In another embodiment, the present invention provides a method of generating a patient-specific cancer diagnostic biomarker profile comprising obtaining a serum-containing biological sample from a patient, performing an assay to determine the presence or absence of cancer specific diagnostic biomarkers in the biological sample, and generating a patient-specific cancer diagnostic biomarker profile of the diagnostic biomarkers present in the sample. The assay is performed as described hereinabove.

The results of the assay provide a cancer diagnostic biomarker profile for the patient that is useful to diagnose cancer and optimize a treatment regimen for any particular cancer subtype.

In another embodiment, the present invention provides a method of identifying a subject at risk for developing cancer comprising obtaining an immunoglobulin-containing biological sample from the subject, performing an assay to determine the presence or absence of one or more cancer diagnostic biomarkers in the biological sample, and identifying the subject as at risk for developing cancer if one or more of the cancer diagnostic biomarkers is present. The assay is performed as described herein above.

In yet another embodiment, the present invention provides a substrate on which one or more antigens that are specific for a cancer diagnostic biomarker are immobilized. The present invention also provides, in another embodiment, a microarray comprising a substrate on which one or more antigens that are specifically bound by cancer diagnostic biomarker are immobilized. The substrates and microarrays may be made as described hereinabove and are useful for creating cancer diagnostic biomarker profiles and for the diagnosis of cancer. An antigen may comprise a protein antigen of Table 1, or a polypeptide or peptide fragment thereof containing one or more epitopes recognized by the cancer diagnostic biomarker, or an epitope peptidomimetic that is recognized by the cancer diagnostic biomarker. Peptidomimetics include, for example, D-peptides, peptoids, and β-peptides. The substrate and microarrays may contain, as the antigen, at least one of the protein antigens of Table 1 or fragments thereof containing one or more epitopes recognized by the cancer diagnostic biomarker.

In another embodiment, the substrate and microarrays may contain, as the antigen, at least one of the protein antigens of Table 1 or a polypeptide or peptide fragment thereof containing one or more epitopes recognized by the cancer diagnostic biomarker, or an epitope peptidomimetic that is recognized by the cancer diagnostic biomarker. Peptidomimetics include, for example, D-peptides, peptoids, and β-peptides. In another preferred embodiment of the present invention, the substrate and microarrays contain at least two, or at least three, or at least four, or at least five, or at least six, or at least seven, or at least eight, or at least nine, or all of the protein antigens of Table 1 or polypeptides or peptide fragments thereof containing one or more epitopes recognized by the cancer diagnostic biomarker, or epitope peptidomimetics that are recognized by the cancer diagnostic biomarkers of Table 1.

In a further embodiment, the present invention provides a kit for detecting cancer-specific antibodies in a sample. A kit comprises one or more antigens that are specific for a cancer diagnostic biomarker and means for determining binding of the antigen to a cancer diagnostic biomarker in the sample. The kit may also comprise packaging material comprising a label that indicates that the one or more antigens of the kit can be used for the identification of cancer. Other components such as buffers, controls, detection reagents, and the like known to those of ordinary skill in art may be included in such the kits. The kits are useful for detecting cancer diagnostic biomarkers and for diagnosing cancer.

In one aspect of the invention, after binding of the surface-bound target proteins to autoantibodies present in human sera, the location of antigen-antibody complexes is revealed by probing arrays with anti-human antibodies containing a fluorescent moiety, such as Alexa-Fluor of fluorescein isothiocyanate (FITC). Detection and quantitation of individual spot fluorescence will be carried out using a Genepix 4000B slide reader and its associated softwares. Results of this detection will be analyzed as described in detail below.

In another aspect of the present invention, diagnostic protein microarrays are described that target protein arrays constructed by immobilizing large numbers of purified proteins and used to assay a wide range of biochemical functions, such as protein-protein, protein-DNA, protein-small molecule interactions, enzyme activity, and to detect antibodies and demonstrate their specificity.

In yet another aspect of the invention, proteins that are available in pure form are spotted, regardless of the method of production and as long as it is solubilized in an appropriate spotting buffer system with considerations taken for retention of 3D conformation, attachment to the substratum and reactivity with the antibody are assessed (see below for details). In another embodiment, protein microarrays are produced through in situ cell-free synthesis directly from corresponding DNA arrays linked to surface of the microarray (as described in Stoevesandt et al., 2009).

Those of ordinary skill in the art appreciate that the present novel method for production of protein arrays in situ avoids the need to express and purify the proteins, spotting instead the DNA templates from which proteins are expressed on the array itself from cell free transcription/translation systems. Although, using the spotting of purified proteins for our microarrays may initially be employed, yet, using one or both of these approaches is contemplated.

In another aspect of the invention, construction of arrays are described from sources of proteins such as (1) cell-based expression systems for recombinant proteins, (2) purification from natural sources, and (3) production in vitro by cell-free translation systems. In at least one embodiment, all of these methods are automated for high throughput production.

External Protein Production and Purification

Protein production and purification—by external means—can be carried out by one or more of the following protocols:

Expression in Bacteria (E. coli)
Venkataraman V, Duda T, Ravichandran S, Sharma R K. (2008) Neurocalcin delta Modulation of ROS-GC1, a New Model of Ca2+ Signaling. Biochemistry, 47 (25), 6590-6601

Expression in Yeast (Pichia pastoris)
Li P, Anumanthan A, Gao X G, Ilangovan K, Suzara V V, Düzgünes N, Renugopalakrishnan V. (2007) Expression of recombinant proteins in Pichia pastoris. Appl Biochem Biotechnol. 142:105-24.

Expression in Insect Cells (Sf9)
Alltmann F, Staudacher E, Wilson I B, März L. (1999) Insect cells as hosts for the expression of recombinant glycoproteins. Glycoconj J. 16:109-123.

Expression in Mammalian Cells (COS7, CHO)
Spampinato S, Baiula M, Calienni M. (2007) Agonist-regulated internalization and desensitization of the human nociceptin receptor expressed in CHO cells. Curr Drug Targets. 8:137-146.

In Situ Protein Production

The following methods can also be used simultaneously express and immobilize many proteins in parallel on the chip surface, which avoids the laborious and often costly processes of DNA cloning, expression and separate protein purification are avoided.

PISA Method

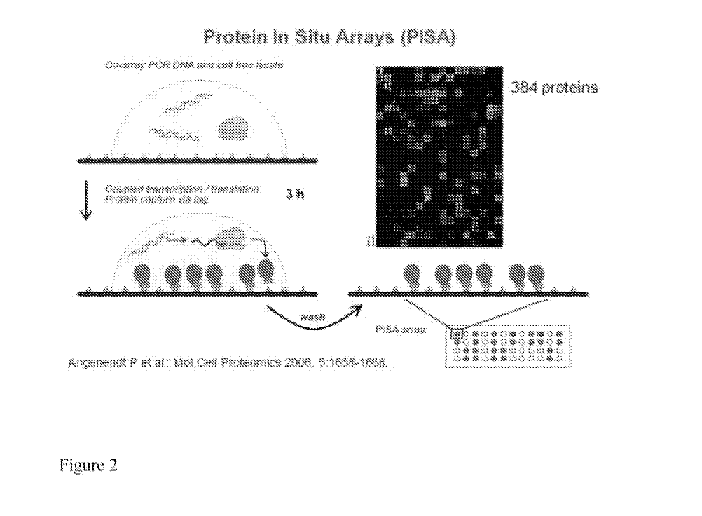

In PISA (He, M., Taussig M. J. Nucleic Acids Res 29, e73, 2001), proteins are made directly from DNA, either in solution or immobilized, and become attached to the array surface as they are made through recognition of a tag sequence. The proteins are expressed in parallel in vitro utilizing a cell free system, commonly rabbit reticulocyte or E. coli S30, to perform coupled transcription and translation. The key feature of the method is that protein expression is performed on a surface which is precoated with an immobilizing agent capable of binding to the tag. Thus after each protein is translated, it becomes fixed simultaneously and specifically to the adjacent surface, while the other materials can subsequently be washed away. Starting from PCR DNA, the PISA procedure will take about 3-4 hours to create the protein array. Microarrays are produced directly onto glass slides, either by mixing the DNA with the cell free lysate system before spotting or by a multiple spotting technique (MIST) in which DNA is spotted first followed by the expression system (Angenendt P. et al, 2006). A schematic is shown in FIG. 2.

NAPPA Method

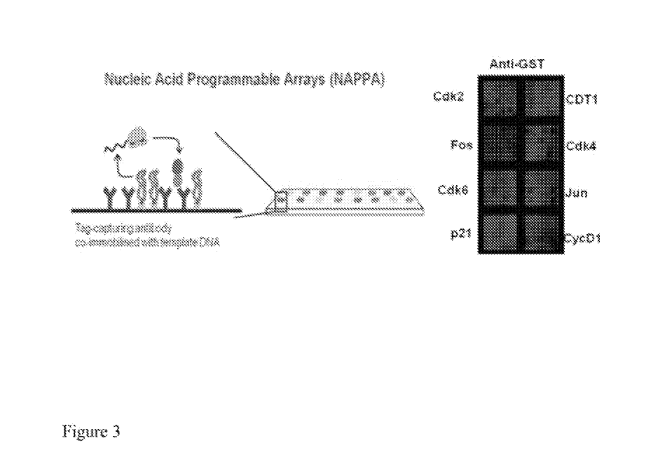

Transcription and translation from an immobilized (as opposed to a solution) DNA template is a further desirable development of on-chip technologies which would allow conversion of DNA arrays to protein arrays. This has been initiated with the system called Nucleic Acid Programmable Protein Array or NAPPA (Ramachandran N. et al. Science 305, 86-90, 2004). Biotinylated cDNA plasmids encoding the proteins as GST fusions are printed onto an avidin-coated slide, together with an anti-GST antibody acting as the capture entity. The cDNA array is then covered with rabbit reticulocyte lysate to express the proteins, which become trapped by the antibody adjacent to each DNA spot, the proteins thereby becoming immobilized with the same layout as the cDNA. This procedure has been shown to work quite precisely, with discrete protein spots and minimal diffusion or cross-talk, and has been used for functional studies of interactions between cell cycle proteins. Recently it was expanded to high density arrays of 1000 different proteins (Ramachandran et al. Nature Methods 5:535-538, 2008). Note, that this technology generates a protein array in which the immobilized proteins are present together with DNA and capture agent, and furthermore that the DNA array can only be used once. A schematic is shown in FIG. 3.

DAPA Method

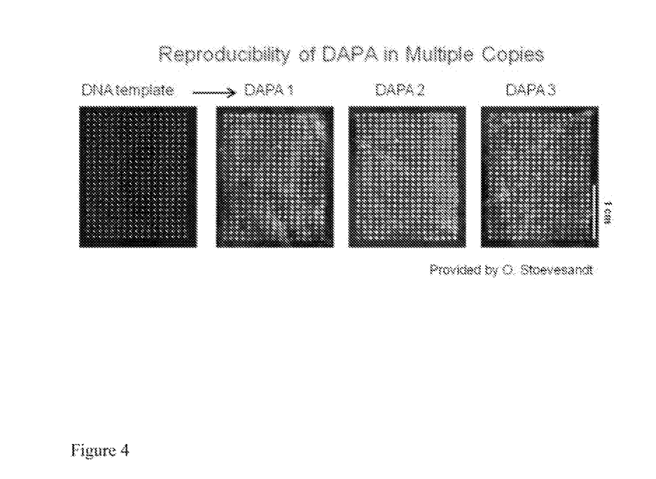

This method for in situ protein arraying represents a further advance in that it uses an immobilized DNA array as the template to generate ‘pure’ protein arrays on a separate surface from the DNA, and also is able to produce multiple copies of a protein array from the same DNA template (He M, et al. Nature Methods, 5, 175-7, 2008). Cell-free protein synthesis is performed in a membrane held between two surfaces (glass slides), one of which is arrayed with DNA molecules while the other surface carries a specific reagent to capture the translated proteins. Individual, tagged proteins are synthesized in parallel from the arrayed DNA, diffuse across the gap and are subsequently immobilized through interaction with the tag-capturing reagent on the opposite surface to form a protein array. Discrete spots which accurately reflect the DNA in position and quantity are produced. Moreover replicate copies of the protein array can be obtained by reuse of the DNA, and at least repeats have been demonstrated. This is shown in FIG. 4.

Protein Spotting or Printing on a Slide or Other Suitable Substratum

Purified proteins and/or antibodies will be either spotted onto the microarray substratum using a flexible protein microarray inkjet printing system (e.g., ArrayJet—www.arrayjet.co.uk), which enables high quality protein microarray production) or target proteins will be generated on site by attached expression clones. Of course, our goal will be to generate precise rows and columns of proteins which will be converted to colored spots denoting both the presence and amount (via the intensity of color) of serum autoantibodies that have bound within the consistent reaction timeframe and under precisely controlled conditions.

Printing Buffers

Because proteins are more heterogeneous than DNA and sometimes show great variations in solubility, the production of protein microarrays will require the use of special (but commercially available) printing buffers. These buffers are important because they are designed to have the spotted protein retain as much of its natural 3D shape as possible, which is especially important for antigen-antibody interactions as they require precise surface matching. The printing buffers can vary somewhat in viscosity (usually due to their content of glycerol, which improves protein stability), and they can also be kept at low temperatures (down to 6° C.) during printing, which further promotes protein stability. An increase in viscosity or decrease in temperature does not affect the spotting of the protein if using commercial inkjet printers such as the ArrayJet Sprint.

The Printing Substratum

A number of different substrata are already available for use, and the one best suited for our purposes will be determined empirically. Currently, our first choice is nitrocellulose-coated glass slides—proteins can attach covalently to the nitrocellulose yet retain their ability to interact specifically with other proteins such as antibodies.

Protein Immobilization on the Substratum

Variables in the immobilization of proteins on the microarray surface include both the coupling reagent and the nature of the surface being coupled to. The properties of a good protein array support surface are that it should be chemically stable before and after the coupling procedures; allow good spot morphology; display minimal nonspecific binding; not contribute to background in detection systems; and be compatible with different detection systems. The immobilization method used should be reproducible; applicable to proteins of different properties (size, hydrophilic, and hydrophobic); amenable to high throughput and automation; and compatible with retention of protein function. The specific orientation of the surface-bound protein is a potentially important feature for antibody microarrays; the most efficient binding results have been obtained with orientated capture reagents (e.g., antibodies), which generally require site-specific labeling of the protein.

Both covalent and noncovalent methods of protein immobilization are available and have various pros and cons. Diffusion of applied proteins into a porous surface (e.g., a hydrogel) is a successful method, allowing noncovalent binding of unmodified protein within hydrogel structures—e.g., a 3-dimensional polyacrylamide gel.

These substrates have been found to give a particularly low background on glass microarrays, with a high capacity and retention of protein function. Passive adsorption of proteins to these surfaces is methodologically simple, but allows little quantitative or orientation control; it may alter the functional properties of the protein through unfolding, and reproducibility and efficiency are variable. Covalent coupling methods provide a stable linkage, can be applied to a range of proteins and have good reproducibility; however, orientation may be variable, and chemical derivatization may alter the function of the protein. Biological capture methods utilising a tag (e.g., hexahistidine/Ni-NTA or biotin/avidin) on the protein provide a stable linkage and bind the protein specifically and in reproducible orientation, but the partner reagent must first be immobilized adequately on the surface.

Array Fabrication

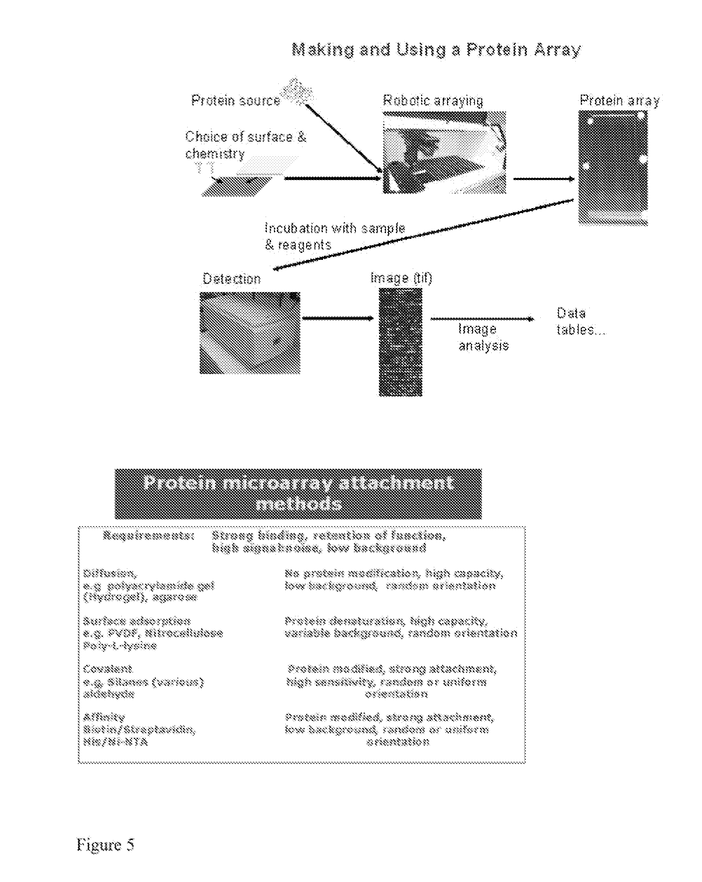

Array fabrication methods include robotic contact printing, ink-jetting, piezoelectric spotting and photolithography. A schematic is shown in FIG. 5. A number of commercial arrayers (spotters) are available as well as manual equipment. The novel methods for production of protein arrays in situ (see section below) avoid the need to express and purify the proteins, spotting instead the DNA templates from which proteins are expressed on the array itself from cell free transcription/translation systems.

As mentioned above, one device that will be used for inkjet printing will be an Arrayjet printer or comparable device. The ArrayJet printer has a stainless steel device called a JetSpyder, which docks with the printhead, is flushed in preparation for sampling and then moves to the wells of the microtiter plate containing samples to be arrayed. Antigen target protein samples in volumes of 5 uL and below in microtiter plate wells are drawn into the capillaries of the JetSpyder, and from here is aspirated into the channels of the printhead. The Arrayjet printhead is a piezoelectric inkjet printhead (Xaar XJ 126) that has been adapted by Arrayjet to this purpose. The print head is suitable for printing nucleic acids, proteins, cell lysates, carbohydrates, blood, nanoparticles and some organic chemistry, onto a range of substrates, including aminosilane, poly-L-lysine, epoxy silane, nitrocellulose, aldehyde, silicon nitride, and gold surfaces.

The printhead contains 126 nozzles in a linear arrangement, and can print can print viscous samples up to 20 centipoise (cP) as well as complex mixtures such as cell lysates. Protein samples are dispensed between the print head and the substrate “on the fly” (no direct contact with substratum). This is particularly important if using potentially fragile microarray substrates. Precise volume deposition (100 pL) rather than passive transfer via capillary action has been found to yield the most appropriate and reproducible spot morphology (round and 90-120 microns in diameter) and eliminates printing artifacts that could compromise data quality in downstream analysis. It will also be possible to have the print head deposit several drops of a given sample in the same feature location, for example, to increase spot size or sample concentration on the fly and within a given print run. Lastly, because the protein spots are so small, it is possible to put 3 duplicate microarrays on the same slide that will be exposed to the same conditions.

With regards to cross- and carry-over contamination and in view of the tendency of proteins to bind indiscriminately to essentially any given surface, we will use a number of printhead and JetSpyder cleaning steps that have been found to adequately address this potential problem. In the microarray printing field, the inkjet spots show better spot morphology as well as reduced diameter variance and less intra-slide concentration variation. In addition, in the future it is possible that CMOS chips are being used as the substratum for protein microarrays, which would allow the detection method to be purely electronic, and for the electronic feed to be captured and processed immediately into images and data Tables.

A number of technical challenges in protein array printing have already be addressed, including sample integrity, viscosity, and variability, substrate surface integrity, and elimination of cross- and carry-over contamination. In my opinion, these challenges have been most effectively addressed by Arrayjet's inkjet microarray spotters as well as a few other companied marketing protein microarray printing systems, making them well suited for high-quality protein microarray production.

Procuring Serum from Patient

To obtain serum from blood, a sample of blood as small as a few drops (such as that obtained via a single finger stick or small tube of blood) will be received and centrifuged at a speed sufficient to pellet all cells and platelets, and the serum to be analyzed will be drawn from the resulting supernatant. To standardize the analysis and to facilitate direct comparison of one serum sample with another or with a reference standard, a precise volume of each serum sample will be exactly and reproducibly diluted (see below) with a suitable buffer (see below) before being applied to our protein microarray containing adherent candidate, antigen targets.

Various cancers can be detected or diagnosed using a multi-diagnostic protein microarray described in this application. Examples of the cancers include Adenoid Cystic Carcinoma, Adrenal Gland Tumor, Amyloidosis, Anal Cancer, Appendix Cancer, Astrocytoma—Childhood, Ataxia-Telangiectasia, Attenuated Familial Adenomatous Polyposis, Beckwith-Wiedemann Syndrome, Bile Duct Cancer, Birt-Hogg-Dube Syndrome, Bladder Cancer, Bone Cancer, Brain Stem Glioma—Childhood, Brain Tumor, Breast Cancer, Breast Cancer—Inflammatory, Breast Cancer—Male, Breast Cancer—Metaplastic, Carcinoid Tumor, Carney Complex, Central Nervous System—Childhood, Cervical Cancer, Childhood Cancer, Colorectal Cancer, Cowden Syndrome, Craniopharyngioma—Childhood, Desmoplastic Infantile Ganglioglioma—Childhood, Endocrine Tumor, Ependymoma, Childhood, Esophageal Cancer, Ewings Family of Tumors, Childhood, Eye Cancer, Eyelid Cancer, Fallopian Tube Cancer, Familial Adenomatous Polyposis, Familial Malignant Melanoma, Gallbladder Cancer, Gardner Syndrome, Gastrointestinal Stromal Tumor—GIST, Germ Cell Tumor—Childhood, Gestational Trophoblastic Tumor, Head and Neck Cancer, Hereditary Breast and Ovarian Cancer, Hereditary Diffuse Gastric Cancer, Hereditary Leiomyomatosis and Renal Cell Cancer, Hereditary Mixed Polyposis Syndrome, Hereditary Non-Polyposis Colorectal Cancer, Hereditary Non-VHL Clear Cell Renal Cell Carcinoma, Hereditary Pancreatitis, Hereditary Papillary Renal Cell Carcinoma, HIV and AIDS-Related Cancer, Islet Cell Tumor, Juvenile Polyposis Syndrome, Kidney Cancer, Lacrimal Gland Tumor, Laryngeal and Hypopharyngeal Cancer, Leukemia—Acute Lymphoblastic—ALL—Childhood, Leukemia—Acute Lymphocytic—ALL, Leukemia—Acute Myeloid—AML, Leukemia—Acute Myeloid—AML—Childhood, Leukemia—B-Cell, Leukemia—Chronic Lymphocytic—CLL, Leukemia—Chronic Myeloid—CML, Leukemia—Eosinophilic, Leukemia—T-Cell, Li-Fraumeni Syndrome, Liver Cancer, Lung Cancer, Lymphoma—Hodgkin, Lymphoma—Hodgkin—Childhood, Lymphoma—Non-Hodgkin—Childhood, Lymphoma—Non-Hodgkin, Mastocytosis, Medulloblastoma, Childhood, Melanoma, Meningioma, Mesothelioma, Muir-Torre Syndrome, Multiple Endocrine Neoplasia Type 1, Multiple Endocrine Neoplasia Type 2, Multiple Myeloma, Myelodysplastic Syndromes—MDS, MYH-Associated Polyposis, Nasal Cavity and Paranasal Sinus Cancer, Nasopharyngeal Cancer, Neuroblastoma—Childhood, Neuroendocrine Tumor, Neurofibromatosis Type 1, Neurofibromatosis Type 2, Nevoid Basal Cell Carcinoma Syndrome, Oral and Oropharyngeal Cancer, Osteosarcoma—Childhood, Ovarian Cancer, Pancreatic Cancer, Parathyroid Cancer, Penile Cancer. Peutz-Jeghers Syndrome, Pituitary Gland Tumor, Pleuropulmonary Blastoma—Childhood, Prostate Cancer, Retinoblastoma—Childhood, Rhabdomyosarcoma—Childhood, Salivary Gland Cancer, Sarcoma, Sarcoma—Alveolar Soft Part and Cardiac, Sarcoma—Kaposis, Skin Cancer (Non-Melanoma), Small Bowel Cancer, Stomach Cancer, Testicular Cancer, Thymoma, Thyroid Cancer, Tuberous Sclerosis Syndrome, Turcot Syndrome, Unknown Primary, Uterine Cancer, Vaginal Cancer, Von Hippel-Lindau Syndrome, Vulvar Cancer, Waldenstrom's Macroglobulinemia Werner Syndrome, Wilms Tumor—Childhood, and Xeroderma Pigmentosa.

### EXAMPLES

To demonstrate its potential efficacy with cancer, the inventors included the preliminary breast cancer data below. The procedure, protocol, materials, and results are as follows:

### Procedure

The protein microarray platform used to identify diagnostic antibodies and prove the efficacy of a protein microarray diagnostic was Invitrogen's ProtoArray® Human Protein Microarray v5.0. It is a high-density protein microarray containing thousands of purified human proteins for protein interaction screening. Each human open reading frame (ORF) is expressed as an N-terminal GST fusion protein using a baculovirus expression system, purified from insect cells, and printed in duplicate on a nitrocellulose-coated glass slide. Techniques for making protein microarray are known in the art. See, e.g., Gavin MacBeath et al., Science 289 (5485), 1760-1763; Jones et al., 2006, Nature 439, 168-174; Chen et al., 2006, Curr Opin Chem Biol 10:28-34; Stoevesandt et al., 2009, Expert Rev Proteomics. 6(2): 145-57; Ramachandran et al., Methods Mol. Biol 2006 328:1-14 and He et al., Curr. Opin Biotechnol 19:4-9.

The human proteins spotted on the microarray are expressed in insect cells using an optimized process to maximize the production of soluble recombinant proteins in a high-throughput format (Schweitzer et al., 2003). Proteins are expressed at high levels in insect cells which are similar to mammalian cells with respect to protein folding and post-translational modifications such as phosphorylation and glycosylation (Bouvier et al., 1998; Hollister et al., 2002; Predki, 2003) in contrast to proteins expressed in E. coli. This allows protein interaction detection at a functional level.

The ProtoArray® Human Protein Microarray specifications are listed below:

Total Subarrays: 48 (4 columns×12 rows)

Subarray Size: 4,400 μM×4,400 μM

Subarray Dimensions: 22 rows×22 columns

Median Spot Diameter: ˜110 μM

Spot Center to Center Spacing: 200 μM

Distance Between Subarrays: 100 μM

Replicates per Sample: 2

Total Human Proteins on v5.0 Array: >9,000*
*ProtoArray® Central Portal must be consulted for Lot Specific Information

Although there are many uses for the ProtoArray® protein microarray, the Immune Response Biomarker Profiling application best suited the needs of a diagnostic. All reagents and materials were purchased directly through Invitrogen. The recommended Invitrogen ProtoArray® protocol was strictly adhered to at all times. It is as follows:

Immune Response Biomarker Profiling—Probing Procedure

### Experimental Outline

1. Block the ProtoArray® Human Protein Microarray with Blocking Buffer.
2. Probe the array with diluted (1:500) human serum or plasma.
3. Perform detection using Alexa Fluor® 647 goat anti-human igG.
4. Dry the array for scanning.
5. Scan the array with a fluorescence microarray scanner to obtain an array image.
6. Download the protein array lot specific information from ProtoArray® Central portal and acquire the image data using microarray data acquisition software.
7. Analyze results using ProtoArray® Prospector data analysis software available from www.invitrogen.com/protoarray.

### Materials Needed

1. ProtoArray® Human Protein Microarray v5.0
2. Human serum or plasma sample (dilute the sample 1:500 in Washing Buffer, store on ice until use)
3. Blocking Buffer and Washing Buffer
4. 10× Synthetic Block
5. Alexa Fluor® 647 Goat Anti-Human IgG (Invitrogen Cat. no. A21445)
6. Clean, covered 4-chamber incubation tray, chilled on ice
7. Forceps and deionized water
8. Shaker (capable of circular shaking at 50 rpm, place the shaker at 4° C.)
9. Microarray slide holder and centrifuge equipped with a plate holder.

### Sample Preparation

The IRBP application has been optimized for use with human serum and plasma samples (fresh or frozen). repeated freeze-thaw cycles with samples must be avoided. Prior to use, the sample must be processed to remove any aggregates by centrifugation (12,000×g for 30 seconds on a microcentrifuge), if necessary, it is recommended to using a 1:500 dilution of the serum or plasma sample in Washing Buffer to maximize signals while minimizing false positive and false negative results. Those of ordinary skill in the art can determine whether to optimize the sample dilution to obtain optimal performance.

### Preparing Blocking Buffer

Blocking Buffer (use 5 mL buffer per microarray) pH 7.5, contains 50 mM HEPES, 200 mM NaCl, 0.08% TritonR X-100, 25% Glycerol, 20 mM Reduced glutathione, 1× Synthetic Block, and 1 mM DTT. 100 mL Blocking Buffer fresh can be prepared by mixing the following reagents: 1 M HEPES, pH 7.5 5 mL, 5 M NaCl 4 mL, 10% TritonR X-100 800 μL, 50% Glycerol 50 mL, Reduced glutathione 610 mg, 10× Synthetic Block 10 mL, and Deionized water to 100 mL. The reagents were mixed and adjusted pH to 7.5 with NaOH and added 100 μL of 1 M DTT prior to use. The buffer should be used immediately or the mixture was stored in any remaining buffer at 4° C. for <24 hours.

### Preparing Washing Buffer

The Washing Buffer (use 60 mL butter per microarray) contains 1×PBS, 0.1% Tween 20, and 1× Synthetic Block. Washing Buffer fresh (1.000 mL) can be prepared as follows: (i) mixing 10×PBS 100 mL, 10% Tween 20 10 mL, 10× Synthetic Block 100 mL, and Deionized water to 1,000 mL; and (ii) cooling the mixed reagents to 4° C. The buffer should be used immediately or the mixture was stored in any remaining buffer at 4° C. for <24 hours.

### Probing Procedure

Before starting the probing procedure, make sure that all items are on hand especially buffers, serum or plasma sample diluted in Washing Buffer, and incubation tray. Make sure the buffers are cold and stored on ice until use. Place an incubation tray on ice to chill until use.

Blocking Step

Instructions for blocking the microarray are described below:
1. Immediately place the mailer containing the ProtoArrayR Human Protein Microarray v5.0 at 4° C. upon removal from storage at −20° C. and equilibrate the mailer at 4° C. for at least 15 minutes prior to use.
2. Place ProtoArrayR Human Protein Microarrays with the barcode facing up in the bottom of a 4-chamber incubation tray such that the barcode end of the microarray is near the tray end containing an indented numeral. The indent in the tray bottom is used as the site for buffer removal.
3. Using a sterile pipette, add 5 mL Blocking Buffer into each chamber. Avoid pipetting buffer directly onto the array surface.
4. Incubate the tray for 1 hour at 4° C. on a shaker set at 50 rpm (circular shaking).
5. After incubation, aspirate Blocking Buffer by vacuum or with a pipette. Position the tip of the aspirator or pipette into the indented numeral and aspirate the buffer from each well. Tilt the tray so that any remaining buffer accumulates at the end of the tray with the indented numeral. Aspirate the accumulated buffer. It is important that do not position the tip or aspirate from the microarray surface as this can cause scratches. Immediately proceed to adding the next solution to prevent any part of the array surface from drying which may produce high or uneven background.
6. Proceed immediately to Probing the Array.

Probing the Array
1. Add 5 mL. Washing Buffer at the indented numeral end of the 4-chamber incubation tray without touching the array surface. Incubate the tray for 5 minutes at 4° C. on a shaker set at 50 rpm (circular shaking).
2. Aspirate the buffer using vacuum or pipette as described on Step 5.
3. Add 5 mL serum or plasma diluted (1:500) in Washing Buffer at the indented numeral end of the 4-chamber incubation tray without touching the array surface. Allow the sample to flow across the array surface. Avoid pipetting sample directly onto the array surface.
4. Incubate the tray for 90 minutes at 4° C. on a shaker set at 50 rpm (circular shaking).
5. Aspirate the sample using vacuum or pipette as described on Step 5.
6. Wash each array with 5 mL Washing Buffer with gentle shaking on a shaker set at 50 rpm for 5 minutes at room temperature. Aspirate the Washing Buffer as described on Step 5.
7. Repeat Step 6 four more times using fresh Washing Buffer each time to obtain a total of 5 wash steps.
8. During the wash steps, mix 2.5 μL Alexa FluorR 647 goat anti-human IgG antibody with 5 mL Washing Buffer per array to obtain a final antibody concentration of 1 g/mL. Store on ice until use.
9. Add 5 mL Alexa FluorR 647 antibody solution from Step 8 to the incubation tray at the indented numeral end of the tray without touching the array surface. Allow the solution to flow across the array surface. Avoid pipetting solution directly onto the array surface.
10. Incubate the tray for 90 minutes at 4° C. on a shaker set at 50 rpm (circular shaking).
11. Aspirate the antibody solution as described on Step 5.
12. Wash each array with 5 mL Washing Buffer with gentle shaking on a shaker set at 50 rpm for 5 minutes at room temperature. Aspirate the Washing Buffer as described on Step 5.
13. Repeat Step 12 four more times using fresh Washing Buffer each time to obtain a total of 5 wash steps.
14. Proceed immediately to Drying the Array.

Drying the Array
1. To remove the array from the 4-chamber incubation tray, insert the tip of forceps into the indented numeral end and gently pry the array upward. Using a gloved hand, pick up the microarray by holding the array by its edges.
2. Place the array in a slide holder (or a sterile 50 mL conical tube, if you do not have a slide holder). Ensure the array is properly placed and is secure in the holder to prevent any damage to the array during centrifugation. Briefly dip the slide holder containing the arrays into room temperature distilled water three times to remove salts. If you are not using a slide holder, dip the array into a 50 mL conical tube filled with room temperature distilled water three times.
3. Centrifuge the array in the slide holder or 50 mL conical tube at 200×g for 1 minute in a centrifuge (equipped with a plate rotor, if you are using the slide holder) at room temperature. Verify the array is completely dry.
4. After drying, store the arrays vertically or horizontally in a slide box protected from light. Avoid prolonged exposure to light. To obtain the best results, scan the array within 24 hours of probing.

### Microarray Scanning

The protein microarrays were scanned using the recommended Axon Genepix 4000b imager. Individual slides were inserted into the imager and then scanned using 100% laser power, 635 nm excitation wavelength, PMT 600, and 5 um pixel size. Data was extracted from the image by syncing it with a Genepix Array List (.GAL) file obtained from Invitrogen. GAL files describe the location and identity of all spots on the protein microarray and are used by Genepix Pro software (by Molecular Devices) to generate files that contain pixel intensity information for all features on the array. Genepix Pro then creates a .GPR (Genepix Pixel Results) file that lists all of the pixel intensity data for each protein spot on the microarray in text-only spreadsheet format. It is the GPR file that is imported into Prospector for data analysis.

### Normalization

After acquiring the individual microarray data by scanning the microarrays with an Axon Genepix 4000b imager and performing the initial quantification with Genepix Pro software, the resulting data must be normalized so as to allow microarray-to-microarray comparison. For this, we relied on Invitrogen's proprietary software, Prospector; more specifically, the Immune Response Biomarker Profiling Toolbox application. Each microarray's gpr file was imported into the program, analyzed, and normalized to a linear model.

Fitting the data to a linear model is performed through a robust regression by means of an iteratively re-weighted least-square procedure with an M-estimator, like the median. The linear model uses log-transformed signals to estimate and correct the variations. For each spot replicate r (=1, 2) of protein feature k (=1, . . . , nf) in sub-array j (=1, . . . , 48) on slide i (=1, . . . , ns,) we will fit the following model:

yᵢⱼₖᵣ=αᵢ+βⱼ+τₖ+εᵢⱼₖᵣ

where yijkr is the observed signal in log 2 scale, α is the slide effect, βj is the sub-array/block effect (including printing pin effect), τk is the “true” signal of the protein feature (different protein content printed in different concentration), and εijkr is the error, assuming εijkr˜N(0,σ2). After the coefficients of these effects are estimated using control proteins, the normalized signal in its original scale for each spot is calculated as:

Sᵢⱼₖᵣ=2̂(yᵢⱼₖᵣ−αᵢ−βⱼ)

After normalization, the microarray data is fully adjusted for error and individual variation; formal analysis can begin. It is this adjusted data from which diagnostic significance can be determined.

### Data Analysis

There are multiple accepted methods of determining the diagnostic significance of microarray fluorescence data. To ensure the reproducibility and accuracy of our results, we chose to analyze our data three separate times using three independent and distinct methods. The methods chosen are among the most reliable and consistent available, and are commonly used in similar studies. They are: M-Statistical Prevalence, Nearest Shrunken Centroid Analysis, and Random Forest Decision-Making Trees. To harness each of these unbiased statistical quantification schemes, we utilized Prospector, PAM, and R's Random Forest, respectively. Each of these programs evaluated the protein microarray data to determine which proteins were most significant to diagnose Breast Cancer. The lists reflected one another almost exactly—lending great credence to the overall concept and the idea that protein microarrays can be used as a successful diagnostic. What follows is a brief description of the statistical methods, programs involved, and results generated.

### M-Statistical Analysis

As well as interpreting and normalizing the raw fluorescence data generated by Genepix Pro, Prospector is capable of generating M-Statistics that can, in turn, be used to evaluate each protein's diagnostic significance. Briefly, M-statistics are used to determine the number of assays in one group (e.g. Breast Cancer or Control) that have a signal value for a protein greater than the highest observed signal value of this probe in the comparison group. The M order statistic for the group y of size ny compared to group x of size nx is given by the formula:

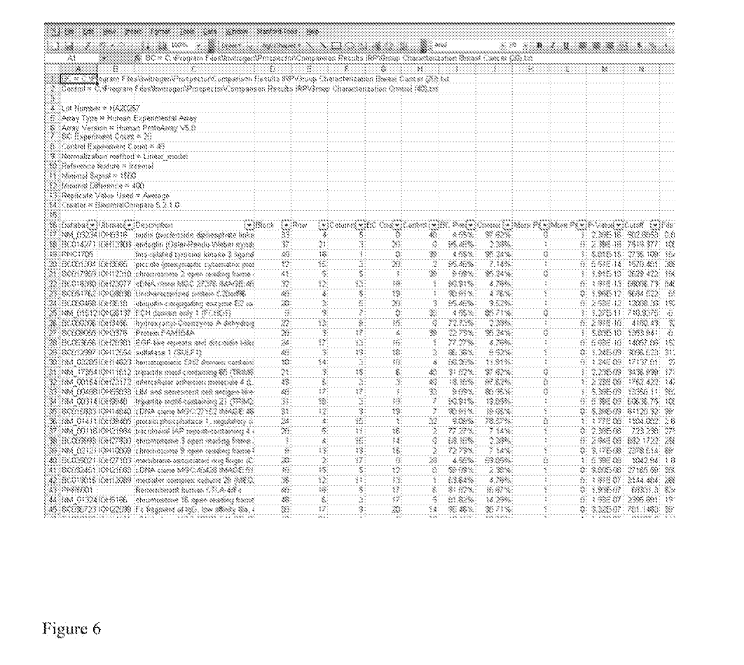

M^{y}_{i,above,between}=Σ1_{{yk>x(i)+between}}1_{{yk>above}}

where x(i) is the ith largest value of the group x, and above and between are the calculation parameters. A p-value is calculated as the probability of having M value greater or equal than Mi. Prospector selects the M statistic with the lowest p-value and reports this Mmax value and order, as well as a corresponding p-value and protein prevalence estimate. When viewed as a spreadsheet in Microsoft Excel Workbook format, these new values can be filtered to provide a list of the most effective indicators of group differences, or, in other words, the proteins that are the best diagnostic markers. FIG. 6 shows a segment of the resulting list of diagnostic proteins:

### PAM (Prediction Analysis of Microarrays)

Another common method of interpreting protein microarray results and yielding protein significance is PAM, or Prediction Analysis of Microarrays. PAM is a statistical technique for class prediction that uses nearest shrunken centroids. It is run as a Microsoft Excel Macro and has been used extensively in characterizing microarray results (Tibshirani, Hastie, Narasimhan, and Chu (2002)). It has also had success in many other classification and survival analysis problems. The program identifies specific subsets of fluorescence data that best characterize each class—and thus would serve as significant diagnostic indicators. Briefly, the method computes a standardized centroid for each class. This is the average fluorescence for protein in each class divided by the within-class standard deviation for that protein. Centroids are “shrunken”—reduced by a threshold value—to reduce error and outlier effect. The microarray fluorescence of each new sample is then compared to each shrunken class centroid; the class whose centroid that it is closest to, in squared distance, is the predicted class for that new sample. Using this information, PAM generates a list of proteins presented in order of diagnostic significance.

With our data, PAM produced the list shown in Table 1 of the top fifty most important proteins for distinguishing Breast Cancer sera from Control Sera:

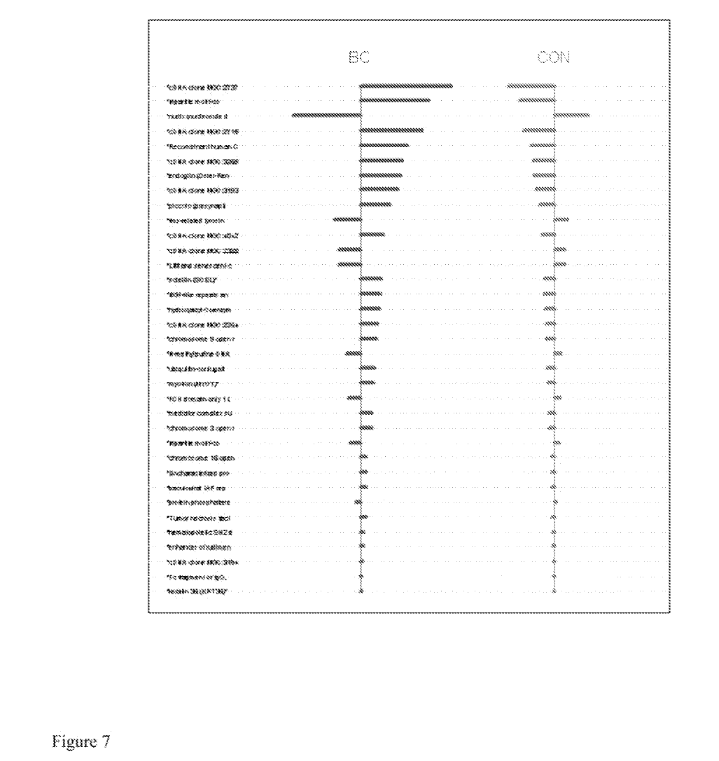

FIG. 7 shows a graphical representation of the PAM significance list. The diagnostic importance of each protein is ordered by descending absolute value difference in presence between Breast Cancer and Control.

### Random Forest

The third and final quantitative method we used to corroborate our results was Random Forest. This is an open-source classification algorithm, run through R, that uses an ensemble of decision-making trees. Each of these classification trees is built using a bootstrap sample of the data, and at each split the candidate set of variables is a random subset. Random Forest directly returns several measures of variable significance, which are related to the relevance of the variable in the classification. Hence, in our case, it provides an evaluation of each protein's relative importance to proper diagnosis.

The most reliable measure is based on the decrease of classification accuracy when values of a variable in a node of a tree are permuted randomly and this is the measure of variable importance. Another estimation of significance of a variable is based on Gini impurity. Every time a split of a node is made on variable m the Gini impurity criterion for the two descendent nodes is less than the parent node. Adding up the Gini decreases for each individual variable over all trees in the forest gives a fast variable importance that is often very consistent with the permutation importance measure.

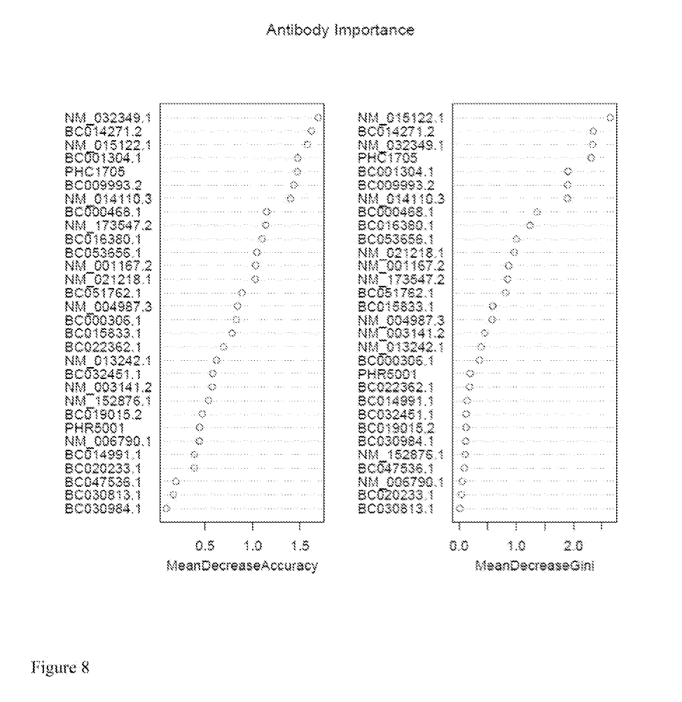

We imported the Relative Fluorescence Unit value for each protein spot on the microarray, as calculated by Genepix Pro and Prospector, into Random Forest. The prediction model was performed using the R package and all default settings—as is proscribed for the best microarray analysis results. Calculating an average Out-Of-Bag Error 0.00%, the algorithm was able to quickly evaluate protein significance based on the evaluation methods described above: the results are shown in FIG. 8.

### Results

From the analysis as outlined above, three conclusions can be drawn.

(1) It is possible to use serum autoantibodies and a protein microarray platform to diagnose Breast Cancer.

(2) There are many autoantibodies that have the capacity to be diagnostic indicators. We used three different, unbiased statistical methods to evaluate the diagnostic significance of individual autoantibodies in our microarray data and they reflected one another almost perfectly. The three resultant lists considered the same autoantibodies diagnostically important, and assigned them similar significance. It would have been possible to identify diagnostic indicators using any one of the methods outlined above, but the shared conclusions of all three lend our results great confidence.

(3) Using even a small subset of the identified significant indicators, it is possible to diagnose breast cancer with great accuracy. To exemplify the diagnostic power of our results, we used only fifteen fluorescence values on our protein microarray to classify blinded Random Forest samples as either Breast cancer or Control. Although additional refinement of the indicators used will increase the overall diagnostic accuracy of the device, our initial results are as follows:

0.00% Out of Bag Error

| | BC | Control | |
|---|---|---|---|
| BC | 20 | 0 | 20 |
| Control | 0 | 40 | 40 |
| | 20 | 40 | |

Sensitivity - 100.0%
Specificity - 100.0%
Positive Predictive Value - 100.0%
Negative Predictive Value - 100.0%

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

I claim:

1. A method for diagnosing cancer in a subject in need of such diagnosis comprising obtaining an immunoglobulin-containing biological sample from the subject, performing an assay to determine the presence or absence of one or more cancer diagnostic biomarkers in the biological sample, and diagnosing cancer if one or more cancer diagnostic biomarkers are present.

2. The method of claim 1 wherein the assay is performed by contacting the sample with one or more antigens that are specific for a cancer diagnostic biomarker under conditions that allow an immunocomplex of the antigen and the biomarker to form, and detecting the presence or absence of an immunocomplex, wherein the presence of an immunocomplex is indicative of the presence of a cancer diagnostic biomarker and wherein the absence of an immunocomplex is indicative of the absence of a cancer diagnostic biomarker.

3. The method of claim 1 wherein the cancer diagnostic biomarker specifically binds to a protein antigen of Table 1.

4. The method of claim 2 wherein the cancer diagnostic biomarker specifically binds to a protein antigen of Table 1.

5. The method of claim 1 wherein the antigen is a protein antigen of Table 1 or a fragment of said protein antigen that contains one or more epitopes recognized by a cancer diagnostic biomarker.

6. The method of claim 2 wherein the antigen is a protein antigen of Table 1 or a fragment of said protein antigen that contains one or more epitopes recognized by an cancer diagnostic biomarker.

7. The method of claim 1 wherein the immunoglobulin-containing sample is serum, plasma or whole blood.

8. The method of claim 2 wherein the one or more antigens are attached to a substrate.

9. The method of claim 2 wherein the one or more antigens are in the form of an array.

10. The method of claim 9 wherein the array is a microarray.

11. The method of claim 10 wherein the microarray comprises a substrate having the protein antigens of Table 1 immobilized thereon.

12. The method of claim 8 wherein the substrate is a nitrocellulose-coated glass slide.

13. A method of generating a patient-specific cancer diagnostic biomarker profile comprising obtaining an immunoglobulin-containing biological sample from a patient, performing an assay to determine the presence or absence of one or more cancer diagnostic biomarkers in the biological sample, and generating a patient-specific cancer diagnostic biomarker profile of the cancer diagnostic biomarkers present in the sample.

14. The method of claim 13 wherein the assay is performed by contacting the sample with one or more antigens that are specific for a cancer diagnostic biomarker under conditions that allow an immunocomplex of the antigen and the biomarker to form, and detecting the presence or absence of an immunocomplex, wherein the presence of an immunocomplex is indicative of the presence of a cancer diagnostic biomarker and wherein the absence of an immunocomplex is indicative of the absence of a cancer diagnostic biomarker.

15. A method for identifying a subject at risk for developing cancer comprising obtaining an immunoglobulin-containing biological sample from the subject, performing an assay to determine the presence or absence of one or more cancer diagnostic biomarkers in the biological sample, and identifying the subject as at risk for developing cancer if one or more cancer diagnostic biomarkers are present.

16. The method of claim 15 wherein the assay is performed by contacting the sample with one or more antigens that are specific for a cancer diagnostic biomarker under conditions that allow an immunocomplex of the antigen and the biomarker to form, and detecting the presence or absence of an immunocomplex, wherein the presence of an immunocomplex is indicative of the presence of a cancer diagnostic biomarker and wherein the absence of an immunocomplex is indicative of the absence of a cancer diagnostic biomarker.

17. A substrate on which one or more antigens that are specific for a cancer diagnostic biomarker are immobilized.

18. The substrate of claim 17 having immobilized thereon a plurality of individually addressable antigens that are specific for cancer diagnostic biomarkers.

19. The substrate of claim 17 wherein the substrate is a slide or a bead.

20. A microarray comprising a substrate on which one or more antigens that are specific for a diagnostic biomarker are immobilized.

21. The microarray of claim 20 wherein the protein antigens of Table 1 or fragments thereof containing one or more epitopes recognized by a cancer diagnostic biomarker are immobilized on the substrate.

22. The microarray of claim 20 having immobilized thereon one or more antigens that are specific for a cancer selected from the group of Breast, Endometrial, Kidney, Ovarian, Cervical, Testicular, Lung, Prostate, Colorectal, Oral, Stomach, Esophageal, Thyroid, Pancreatic, Bladder Cancer, Adenoid Cystic Carcinoma, Adrenal Gland Tumor, Amyloidosis, Anal Cancer, Appendix Cancer, Astrocytoma—Childhood, Ataxia-Telangiectasia, Attenuated Familial Adenomatous Polyposis, Beckwith-Wiedemann Syndrome, Bile Duct Cancer, Birt-Hogg-Dube Syndrome, Bladder Cancer, Bone Cancer, Brain Stem Glioma—Childhood, Brain Tumor, Breast Cancer, Breast Cancer—Inflammatory, Breast Cancer—Male, Breast Cancer—Metaplastic, Carcinoid Tumor, Carney Complex, Central Nervous System—Childhood, Cervical Cancer, Childhood Cancer, Colorectal Cancer, Cowden Syndrome, Craniopharyngioma—Childhood, Desmoplastic Infantile Ganglioglioma—Childhood, Endocrine Tumor, Ependymoma—Childhood, Esophageal Cancer, Ewings Family of Tumors—Childhood, Eye Cancer, Eyelid Cancer, Fallopian Tube Cancer, Familial Adenomatous Polyposis, Familial Malignant Melanoma, Gallbladder Cancer, Gardner Syndrome, Gastrointestinal Stromal Tumor—GIST, Germ Cell Tumor—Childhood, Gestational Trophoblastic Tumor, Head and Neck Cancer, Hereditary Breast and Ovarian Cancer, Hereditary Diffuse Gastric Cancer, Hereditary Leiomyomatosis and Renal Cell Cancer, Hereditary Mixed Polyposis Syndrome, Hereditary Non-Polyposis Colorectal Cancer, Hereditary Non-VHL Clear Cell Renal Cell Carcinoma, Hereditary Pancreatitis, Hereditary Papillary Renal Cell Carcinoma, HIV and AIDS-Related Cancer, Islet Cell Tumor, Juvenile Polyposis Syndrome, Kidney Cancer, Lacrimal Gland Tumor, Laryngeal and Hypopharyngeal Cancer, Leukemia—Acute Lymphoblastic—ALL—Childhood, Leukemia—Acute Lymphocytic—ALL, Leukemia—Acute Myeloid—AML, Leukemia—Acute Myeloid—AMI—Childhood, Leukemia—B-Cell, Leukemia—Chronic Lymphocytic—CLL, Leukemia—Chronic Myeloid—CML, Leukemia—Eosinophilic, Leukemia—T-Cell, Li-Fraumeni Syndrome, Liver Cancer, Lung Cancer, Lymphoma—Hodgkin, Lymphoma—Hodgkin—Childhood, Lymphoma—Non-Hodgkin—Childhood, Lymphoma—Non-Hodgkin, Mastocytosis, Medulloblastoma—Childhood, Melanoma, Meningioma, Mesothelioma, Muir-Torre Syndrome, Multiple Endocrine Neoplasia Type 1, Multiple Endocrine Neoplasia Type 2, Multiple Myeloma, Myelodysplastic Syndromes—MDS, MYH-Associated Polyposis, Nasal Cavity and Paranasal Sinus Cancer, Nasopharyngeal Cancer, Neuroblastoma—Childhood, Neuroendocrine Tumor, Neurofibromatosis Type 1, Neurofibromatosis Type 2, Nevoid Basal Cell Carcinoma Syndrome, Oral and Oropharyngeal Cancer, Osteosarcoma—Childhood, Ovarian Cancer, Pancreatic Cancer, Parathyroid Cancer, Penile Cancer, Peutz-Jeghers Syndrome, Pituitary Gland Tumor, Pleuropulmonary Blastoma—Childhood, Prostate Cancer—Retinoblastoma—Childhood, Rhabdomyosarcoma—Childhood, Salivary Gland Cancer, Sarcoma, Sarcoma—Alveolar Soft Part and Cardiac, Sarcoma—Kaposis, Skin Cancer (Non-Melanoma), Small Bowel Cancer, Stomach Cancer, Testicular Cancer, Thymoma, Thyroid Cancer, Tuberous Sclerosis Syndrome, Turcot Syndrome, Unknown Primary, Uterine Cancer, Vaginal Cancer, Von Hippel-Lindau Syndrome, Vulvar Cancer, Waldenstrom's Macroglobulinemia, Werner Syndrome, Wilms Tumor—Childhood, and Xeroderma Pigmentosa.

23. A kit comprising one or more antigens that are specific for a cancer diagnostic biomarker and means for determining binding of the antigen to a cancer diagnostic biomarker in an immunoglobulin-containing biological sample, wherein cancer is selected from the group consisting of Breast, Endometrial, Kidney, Ovarian, Cervical, Testicular, Lung, Prostate, Colorectal, Oral, Stomach, Esophageal, Thyroid, Pancreatic, Bladder Cancer, Adenoid Cystic Carcinoma, Adrenal Gland Tumor, Amyloidosis, Anal Cancer, Appendix Cancer, Astrocytoma—Childhood, Ataxia-Telangiectasia, Attenuated Familial Adenomatous Polyposis, Beckwith-Wiedemann Syndrome, Bile Duct Cancer, Birt-Hogg-Dube Syndrome, Bladder Cancer, Bone Cancer, Brain Stem Glioma—Childhood, Brain Tumor, Breast Cancer, Breast Cancer—Inflammatory, Breast Cancer—Male, Breast Cancer—Metaplastic, Carcinoid Tumor, Carney Complex, Central Nervous System—Childhood, Cervical Cancer, Childhood Cancer, Colorectal Cancer, Cowden Syndrome, Craniopharyngioma—Childhood, Desmoplastic Infantile Ganglioglioma—Childhood, Endocrine Tumor, Ependymoma—Childhood, Esophageal Cancer, Ewings Family of Tumors—Childhood, Eye Cancer, Eyelid Cancer, Fallopian Tube Cancer, Familial Adenomatous Polyposis, Familial Malignant Melanoma, Gallbladder Cancer, Gardner Syndrome, Gastrointestinal Stromal Tumor—GIST, Germ Cell Tumor—Childhood, Gestational Trophoblastic Tumor, Head and Neck Cancer, Hereditary Breast and Ovarian Cancer, Hereditary Diffuse Gastric Cancer, Hereditary Leiomyomatosis and Renal Cell Cancer, Hereditary Mixed Polyposis Syndrome, Hereditary Non-Polyposis Colorectal Cancer, Hereditary Non-VHL Clear Cell Renal Cell Carcinoma, Hereditary Pancreatitis, Hereditary Papillary Renal Cell Carcinoma, HIV and AIDS-Related Cancer, Islet Cell Tumor, Juvenile Polyposis Syndrome, Kidney Cancer, Lacrimal Gland Tumor, Laryngeal and Hypopharyngeal Cancer, Leukemia—Acute Lymphoblastic—ALL—Childhood, Leukemia—Acute Lymphocytic—ALL, Leukemia—Acute Myeloid—AML, Leukemia—Acute Myeloid—AML—Childhood, Leukemia—B-Cell, Leukemia—Chronic Lymphocytic—CLL, Leukemia—Chronic Myeloid—CML, Leukemia—Eosinophilic, Leukemia—T-Cell, Li-Fraumeni Syndrome, Liver Cancer, Lung Cancer, Lymphoma—Hodgkin, Lymphoma—Hodgkin—Childhood, Lymphoma—Non-Hodgkin—Childhood, Lymphoma—Non-Hodgkin, Mastocytosis, Medulloblastoma—Childhood, Melanoma, Meningioma, Mesothelioma, Muir-Torre Syndrome, Multiple Endocrine Neoplasia Type 1, Multiple Endocrine Neoplasia Type 2, Multiple Myeloma, Myelodysplastic Syndromes—MDIS, MYH-Associated Polyposis, Nasal Cavity and Paranasal Sinus Cancer, Nasopharyngeal Cancer, Neuroblastoma—Childhood, Neuroendocrine Tumor, Neurofibromatosis Type 1, Neurofibromatosis Type 2, Nevoid Basal Cell Carcinoma Syndrome, Oral and Oropharyngeal Cancer, Osteosarcoma—Childhood, Ovarian Cancer, Pancreatic Cancer, Parathyroid Cancer, Penile Cancer, Peutz-Jeghers Syndrome, Pituitary Gland Tumor, Pleuropulmonary Blastoma—Childhood, Prostate Cancer,—Retinoblastoma—Childhood, Rhabdomyosarcoma—Childhood, Salivary Gland Cancer, Sarcoma, Sarcoma—Alveolar Soft Part and Cardiac, Sarcoma—Kaposis, Skin Cancer (Non-Melanoma), Small Bowel Cancer, Stomach Cancer, Testicular Cancer, Thymoma, Thyroid Cancer, Tuberous Sclerosis Syndrome, Turcot Syndrome, Unknown Primary, Uterine Cancer, Vaginal Cancer, Von Hippel-Lindau Syndrome, Vulvar Cancer, Waldenstrom's Macroglobulinemia, Werner Syndrome, Wilms Tumor—Childhood, and Xeronerma Pigmentosa.

24. The kit of claim 23 wherein the one or more autoantigens are immobilized on a substrate.
